(19) United States
(12) Patent Application Publication
(10) Pub. No.: US 2019/0382785 A1
(43) Pub. Date: Dec. 19, 2019

(54) Development of Herbicide and Sucking Pest Resistant Plant [Kalgin-5] by the Over-Expression of Constitutive Promoters Driven Tetra Gene Construct

(71) Applicant: FB Genetics (Pvt) Limited, Lahore (PK)

(72) Inventors: Daniyal Jawed Qureshi, Lahore Cantt (PK); Hamza Nadeem Qureshi, Lahore Cantt (PK)

(21) Appl. No.: 16/407,926

(22) Filed: May 9, 2019

(30) Foreign Application Priority Data
May 9, 2018 (PK) 334/2018

(51) Int. Cl. C12N 15/82; C07K 14/195; C07K 14/415

(52) U.S. Cl. CPC C12N 15/8274; C07K 14/415; C07K 14/195; C12N 15/8286

(57) ABSTRACT

Recombinant or synthetic polynucleotide sequences comprising three sucking pest resistant genes including an insecticidal Tma12 gene (SEQ ID NO: 18), a PTA gene (SEQ ID NO: 19) and ASAL gene (SEQ ID NO: 20) and a herbicidal Re-PAT gene (SEQ ID NO: 17), transformed in a mono/dicot plant, that are encoded to provide insecticidal and herbicidal toxin proteins in a transgenic plant with constitutively targeted expression, resulting in the decreased resistance development against insecticidal toxins proteins and increased efficacy against the insect mortality, particularly whitefly and jassid. A method or an assay for detecting the presence of transgenic event Kalgin-5 based on the DNA sequence of the recombinant polynucleotide construct inserted into the genome of the transgenic plant and the genomic sequences flanking the insertion site.

### CROSS-REFERENCE TO RELATED APPLICATIONS

This application claims the benefit of Pakistani Application No. PK 334/2018 filed 9 May 2018 the entire contents and substance of which is hereby incorporated by reference.

### SEQUENCE LISTING

The instant application contains a Sequence Listing which has been submitted electronically in ASCII format and is hereby incorporated by reference in its entirety. Said ASCII copy, created on May 9, 2019, is named FBG1_SL.txt and is 38,738 bytes in size.

### BACKGROUND OF THE INVENTION

### 1. Technical Field

The present invention relates to the field of genetic engineering of mono and/or dicot plants, more specifically the invention relates to the enhanced expression of synthetic insecticidal genes including Tma12, PTA and ASAL and herbicidal gene Re-PAT in transgenic plant specifically cotton plant (Gossypium hirsutum). The invention also relates to the plant, plant part, plant seeds and/or plant cells related to event Kalgin-5 and provide nucleotide molecules that are unique to the event and created in connection with insertion of transgenic DNA into the genome of a cotton plant and to assays for detecting the presence of Kalgin-5 event in the transgenic cotton plant sample.

### 2. Description of Related Art

Cotton being a cash crop and an essential source of raw material to the textile enables the textile industry to survive and expand its base. Cotton contributes 1% to GDP and has share of 5.1% in agriculture value additions. Massive decline in production of cotton was observed this year, and to maintain continuous supply to textile industry, raw cotton was imported during July which has increased to 345.363 thousand tonnes compared to last year during same period which was 97.354 thousand tonnes, so a growth of 254.75% was noticed while in value terms it touched to US$588.236 million against US$224.647 million showing a growth of 161.85%. Cotton was sown on a territory of 2917 thousand hectares, demonstrating a decline of 1.5% over a year ago in a region of 2961 thousand hectares. A production of 10.074 million bales of cotton was recorded this year which were 27.8% less than previous year (Economic Survey of Pakistan, 2015-16).

Being the main cash crop of Pakistan, it is threatened by broad and narrow weeds, sucking pest including whitefly, aphid jassids and thrips. The whitefly (Bemisia tabaci) is considered to be one of the most invasive organisms and has been included in the list of the 100 of the world's worst invasive alien species. Naturally, it befalls all through tropical and subtropical regions of the world. However, agricultural products movement is the major reason of its global spreading that's why it has become the most damaging agricultural pest in the world within last 20 years. Whitefly damages the plants including cotton both directly, by sucking cell sap from phloem and other parts too, resulting in stunted growth, early wilting and premature defoliation, finally yields loses, and indirectly by the honey dew it excretes, which promote fungal growth on leaves and fruits surfaces. Additionally, it transmits many plant viruses including begomoviruses, criniviruses, ipomoviruses, torradoviruse and some carlaviruses. Bemisia tabaci is a phloem feeder and most begomoviruses are phloem restricted. For this reason, overexpression of toxins against whitefly under the control of constitutive as wells as phloem specific promoters should be of more value both from biosafety standpoint and effective control. Scientist from diverse field, both from public and private sectors, however apart from being hazardous for the environment 100% yield losses may occur even after multiple pesticide for have been working to identify and implement novel resistance strategies. Worldwide whitefly management has mainly focused conventional pest management practices using insecticide and pesticide sprays, because the development of pesticide resistance in B. tabaci. The most widely B. tabaci applied technology in the history of genetically modified crops is the expression of insecticidal endotoxins of bacterial origin (Bacillus thuringiensis). Worldwide several transgenic crops are grown over vast area including 86% area under transgenic cotton (single-gene) in Pakistan.

It is the indication that by far the most practical approach for the development of insect resistant plants is the use of effective toxins. Pyramiding multiple toxins to interfere with different pathways of the target insects has been looking the most rational approach for developing long term resistance and should be considered to engineer durable B. tabaci resistance. The combined effect of the already and recently investigated toxins against sucking pest should ideally provide broad-spectrum resistance. The present investigation aims to transform cotton seed with multiple genes stacked under single T-DNA to have targeted constitutive expression including phloem to encounter wide range of weeds and sucking insects like B. tabaci and other wide range of sucking pest.

In the Pakistani agriculture, there exists a stipulation to raise a cotton plant shows characters with multiple and accumulative resistance to reduce yield loss due to variety of sucking insect pests. In the present invention the tetra gene cotton plant would reduce the need to apply different chemical and pesticides that might be harmful to other beneficial insects and most importantly to environment. Further, tetra gene with different mode of action would help in delaying sucking pest's resistance to lectin/insecticidal genes, which is prevalent problem in Pakistani agriculture with single gene cotton.

Methods of Removing Weeds from Crop Field

The sunlight and nutrients of plants are being competed between crops and unwanted weeds. This competency leads to often substantial yield loss. Tandem techniques of soil tilling or herbicide application are adopted by farmers to control weeds at their farms conventionally. Burdensome and needs intensive labor, time and money. Herbicide, on the other hand have not any distinguished between plants and weeds plants. Selective herbicides are the only options as for as conservative agriculture system is concerned. Such herbicides do not damage the crops but at the same time are not equally effective against different types of weeds plants. This problem can be solved by the farmers by using herbicide resistant crops, and by this they can remove all types of weeds with single and swift application of non-selective herbicides. It is time, labor and cost saving needing less spraying, labor and less traffic on the filed with lower operating charges.

Glufosinate is broad spectrum non-selective post emergent herbicide. It belongs to organic phosphorous family of herbicides. It inhibits glutamine synthesis (GS) and nitrogen assimilation ability of GS. Inhibition of GS leads to accumulation of ammonia and finally indirect inhibition of photosynthesis and then weeds plant death. Glufosinate-ammonium ensures a high degree of crop safety, as it only affects the parts of the plant where it is applied. Its unique mode of action makes it ideal to be used in rotation with other herbicides to mitigate weed resistance. There are several means for the modification of crops to be tolerant to glufosinate. One approach is to genetically engineer crop plant with Re-PAT a marine bacterium (Rhodococcus sp. strain YM12) gene that yield resistant against glufosinate. The implementation of glufosinate-based crop production system is and will be one of the most significant revolutions in the history of agriculture.

### Methods of Controlling Sucking Pest Infestation in Plants

Tectaria macrodonta is an edible plant named fern has insecticidal fern protein which is toxic to a variety of sucking pest and can protect cotton especially from whitefly with its chitinase activity. Recent studies showed that many plant exhibit insecticidal activity on the sap-sucking hemipteran insects (Sengupta et al., 2010). Insecticidal toxic proteins with insecticidal activity have been found environmentally-acceptable topical insecticides because of their toxicity to the specific target insect pests, and non-toxicity to other plants and beneficial non-target organisms. Lectins derived from diverse plant species have been found to provide effective protection against several insect pest like Tectaria macrodonta, Pinellia ternata agglutinin, Allium satiuvum and others have lectin proteins which are classified into chitin-binding lectins, legume lectins, type-2 ribosome-inactivating lectins and the most important is mannose binding lectins. Mannose-binding lectins are especially important because they confer plant defense against insects (Van Damme, 2008). Lectin toxicity in insects seems to involve the binding of lectins to the brush border membrane vesicle receptors of gut epithelial cells, thereby causing disruption of cell function and mortality. Ferns produce phytoecdysones that severely impair insect development and cause molting abnormalities. On the hand Pinellia ternata agglutinin (PTA) is a traditional Chinese medicinal plant native to China, known as the crow dipper. It is naturally grown in the wild and distributed throughout China and east-Asia (Bensky et al., 2004). Lectins or agglutinins of P. ternata (PTA) had significant insecticidal activities against cotton especially against sucking insects. Mannose-specific Allium sativum leaf agglutinin ASAL revealed high-level resistance against major sap-sucking pests in cotton (Bharathi et al., 2011).

### SUMMARY OF THE INVENTION

An objective of the invention is to develop a recombinant polynucleotide sequences of herbicide tolerant Re-PAT (Rhodococcus sp. strain YM12, marine bacterium) gene (SEQ ID NO: 17), insecticidal sucking pest resistant genes i.e. a Tma12 (Tectaria macrodonta) gene (SEQ ID NO: 18), PTA (Pinellia ternata agglutinin) gene (SEQ ID NO: 19) and ASAL (Allium satiuvum) gene, to transformed into mono and/or dicot plants particularly cotton plants.

Another objective of the invention is to develop a recombinant polynucleotide sequences of herbicide tolerant Re-PAT (Rhodococcus sp. strain YM12, marine bacterium) gene (SEQ ID NO: 17), insecticidal sucking pest resistant genes i.e. a Tma12 (Tectaria macrodonta) gene (SEQ ID NO: 18), PTA (Pinellia ternata agglutinin) gene (SEQ ID NO: 19) and ASAL (Allium satiuvum) gene, with enhanced expression including phloem-targeted expression in the transgenic mono and/or dicot plants specifically cotton plants.

Another objective of the invention is to develop enhanced assembled expression of Re-PAT, Tma12, PTA and ASAL genes to make transgenic tetra gene plant particularly cotton plant, more tolerant and effective in controlling to broad and narrow leave range of weeds and hemiptearn insect and pest families than single gene transgenic plants or cotton plants.

One another objective of the invention is to develop an identification of recombinant polynucleotide sequences identified as SEQ ID NOS: 1-16 and SEQ ID NOS: 27-28 that are useful as primer sequences for the detection of the respective recombinant polynucleotide sequences.

In addition to above, another objective of the present invention is to develop a recombinant polynucleotide sequence identified as SEQ ID NO: 21 to offer a superior strategy for demolishing of insect resistance by enhanced collective expression of herbicidal gene Re-PAT, and insecticidal proteins like Tma12, PTA and ASAL gene within the single T-DNA even all four including three insecticidal genes have not significant homology with each other.

Another aspect of the invention, methods or assays for detecting the presence of the transgene insertion region identified as SEQ ID NO: 26 in transgenic plant specifically in cotton plant.

Further adding aspects includes a method for the enhanced expression of Tma12, PTA, ASAL and Re-PAT proteins conferring resistance against insect pest by expressing them constitutively including phloem of the plants, cotton plant.

In an exemplary embodiment of the present invention, a profusion of cassettes having a recombinant polynucleotide sequences encompasses a tetra gene (SEQ ID NO. 21) with a 5′ end attached by a promoter joined to an un-translated enhancer (intron) sequence and a 3′ end attached to a NOS terminator, for encoding the polynucleotide sequences, wherein the tetra gene comprises sucking pest resistant genes including an insecticidal Tma12 gene (SEQ ID NO. 18) from the fern Tectaria macrodonta, a crow dipper gene PTA (SEQ ID NO. 19) and an Allium sativum gene ASAL (SEQ ID NO. 20) and a Re-PAT gene (SEQ ID NO. 17) are encoded to provide insecticidal and herbicidal toxin proteins in a transgenic plants having constitutively targeted expression, and resulting in the decreased resistance development against insecticidal toxin proteins and increased efficacy against the insect mortality.

A first cassette, a second cassette, a third cassette, and a fourth cassette can be located within a T-DNA region of a vector flanked by a left and right border sequence.

The first cassette coding the insecticidal Tma12 gene having (SEQ ID NO: 18) can be operably linked to the promoter, the un-translated enhancer sequence at a 5′ end and the NOS terminator sequence tagged to a 3′ end of the gene Tma12. The second cassette coding the insecticidal PTA gene having (SEQ ID NO: 19) can be operably linked to the promoter, the un-translated enhancer sequence at a 5′ end and the NOS terminator sequence tagged to a 3′ end of the PTA gene. The third cassette coding the insecticidal ASAL gene having (SEQ ID NO: 20) can be operably linked to the promoter, the un-translated enhancer sequence at a 5′ end and the NOS terminator sequence tagged to a 3′ end of the ASAL gene. The fourth cassette coding Re-PAT herbicidal protein gene having (SEQ ID: 17) can be operably linked to the promoter, the un-translated enhancer sequence at a 5′ end and the NOS terminator sequence tagged to a 3′ end of Re-PAT gene.

The profusion of cassettes having (SEQ ID: 21) can be present in the transgenic plant or the part of the transgenic plant.

The promoter can be the Cauliflower mosaic virus (CaMV35S).

The 5′ end of each gene, for example, the Tma12, the PTA, the ASAL gene and the Re-PAT in the transgenic plant can be attached with the un-translated enhancer sequence comprising 28 nucleotides of SEQ-ID NO. 21 starting from the 7^{th} nucleotide to 34^{th} nucleotide.

The transgenic plant can be a monocot plant selected from the group consisting of maize, sugarcane, and wheat. The transgenic plant can be a dicot plant selected from the group consisting of cotton, potato and tomato.

The profusion of cassettes can be located at a SEQ ID NO. 26 having a forward primer of SEQ ID NO. 27 and a reverse primer of SEQ ID NO. 28 for identification.

In another exemplary embodiment of the present invention, a recombinant DNA molecule can comprise a polynucleotide sequence selected from the group consisting of SEQ ID NOS: 1-21, SEQ ID NO: 26, SEQ ID NO: 27, SEQ ID NO: 28, and complements thereof.

The DNA molecule can comprise SEQ ID NOS: 1-21, SEQ ID NO: 26, SEQ ID NO: 27, and SEQ ID NO: 28, and complements thereof, in the transgenic cotton plant, plant cell, seed or plant part. The DNA molecule can comprise SEQ ID NO: 26 in the transgenic cotton plant, plant cell, seed or plant part. The DNA molecule can comprise SEQ ID NOS: 1-20 in the transgenic cotton plant, plant cell, seed or plant part. The DNA molecule can comprise SEQ ID NOS: 17-20 in the transgenic cotton plant, plant cell, seed or plant part.

In another exemplary embodiment of the present invention, a transgenic cotton plant, seed, cells or plant part can comprise a recombinant DNA molecule comprising a polynucleotide sequence selected from the group consisting of SEQ ID NOS: 1-21, SEQ ID NO: 26, SEQ ID NO: 27, SEQ ID NO: 28, and complements thereof, wherein an amplicon comprises the DNA molecule having the sequence of SEQ ID NOS: 1-16.

In another exemplary embodiment of the present invention, a transgenic cotton plant, seed, cells or plant part can comprise a recombinant DNA molecule comprising a polynucleotide sequence selected from the group consisting of SEQ ID NOS: 1-21, SEQ ID NO: 26, SEQ ID NO: 27, SEQ ID NO: 28, and complements thereof, wherein an amplicon comprises the DNA molecule having the sequence of SEQ ID NO: 21.

### Brief Description of the Sequences

The following nucleotide sequences make part of current narration and are given to further corroborate certain characteristic of the present invention. Reference regarding these sequences may enhance the vision and scope of the present invention and specific embodiments described herein.

### Identification of Sequences

SEQ ID NOS: 1-2 forward and reverse primers to amplify Re-PAT gene.

SEQ ID NOS: 3-4 forward and reverse primers to amplify Tma12 gene.

SEQ ID NOS: 5-6 Forward and reverse primers to amplify PTA gene.

SEQ ID NOS: 7-8 Forward and reverse primers to amplify ASAL gene.

SEQ ID NOS: 9-10 Forward and reverse primers to amplify UTR and Re-PAT gene.

SEQ ID NOS: 11-12 Forward and reverse primers to amplify UTR and Tma12 gene.

SEQ ID NOS: 13-14 Forward and reverse primers to amplify UTR and PTA gene.

SEQ ID NOS: 15-16 Forward and reverse primers to amplify UTR and ASAL gene.

SEQ ID NO: 17 Polynucleotide sequence of Re-PAT gene.

SEQ ID NO: 18 Polynucleotide sequence of Tma12 gene.

SEQ ID NO: 19 Polynucleotide sequence of PTA gene.

SEQ ID NO: 20 Polynucleotide sequence of ASAL gene.

SEQ ID NO: 21 Polynucleotide sequence of T-DNA having all four cassettes.

SEQ ID NO: 22 Polypeptide sequence of Re-PAT precursor protein.

SEQ ID NO: 23 Polypeptide sequence of Tma12 precursor protein.

SEQ ID NO: 24 Polypeptide sequence of PTA precursor protein.

SEQ ID NO: 25 Polypeptide sequence of ASAL precursor protein.

SEQ ID NO: 26 Polynucleotide sequence of synthetic recombinant construct and Gossypium hirsutum genome junction event sequence.

SEQ ID NO: 27 Polynucleotide sequence of primer from synthetic sequence of insert for event detection.

SEQ ID NO: 28 Polynucleotide sequence of primer from Gossypium hirstum genome for event detection.

### DETAILED DESCRIPTION OF THE INVENTION

The following definitions and methods are provided to better define the present invention and to guide those of ordinary skill in the art in the practice of the present invention. Unless otherwise noted, terms are to be understood according to conventional usage by those of ordinary skill in the relevant art.

As used herein, the term “cotton” means Gossypium hirsutum and includes all plant varieties that can be bred with cotton, including wild cotton species.

As used herein, the term “comprising” means “including but not limited to”.

A transgenic “event” is produced by transformation of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular genome location. The term “event” refers to the original transformant and progeny of the transformant that include the heterologous DNA. The term “event” also refers to progeny produced by a sexual outcross between the transformant and another variety that includes the genomic/transgene DNA. Even after repeated back-crossing to a recurrent parent, the inserted transgene DNA and flanking genomic DNA (genomic/transgene DNA) from the transformed parent is present in the progeny of the cross at the same chromosomal location. The term “event” also refers to DNA from the original transformant and progeny thereof comprising the inserted DNA and flanking genomic sequence immediately adjacent to the inserted DNA that would be expected to be transferred to a progeny that receives inserted DNA including the transgene of interest as the result of a sexual cross of one parental line that includes the inserted DNA (e.g., the original transformant and progeny resulting from selfing) and a parental line that does not contain the inserted DNA.

To facilitate an understanding of the principles and features of the various embodiments of the invention, various illustrative embodiments are explained below. Although exemplary embodiments of the invention are explained in detail, it is to be understood that other embodiments are contemplated. The further scope of this present invention will be further highlighted by following content relevant to the present invention, in aggregation with related sequence listing.

The first cassette of present invention provides a G. hirsutum codon optimized purified DNA construct cassette comprising synthetic Tma12 protein encoding region localized to constitutive including phloem or localized to a plant cell nuclear genome and possibly linked to a region encoding constitutive directed sequence which is one means of enabling expression of Tma12 protein in whole cotton plant. The Tma12 gene comprises the sequence of (SEQ ID NO: 18).

In the second cassette, the current invention provides cotton codon relevant optimized DNA construct comprising crow dipper PTA (Pinellia ternata agglutinin) encoding protein constitutively localized or localized to a plant cell nuclear genome and is possibly linked to a region of sequence directed constitutively which is one means of enabling localization of PTA protein to all parts of cotton plant including phloem. The PTA gene comprises the sequence of (SEQ ID NO: 19)

The third one construct cassette of this instant invention comprises Allium sativum agglutinin ASAL gene optimized according to G. hirsutum codons constitutively distributed in cotton plant or localized to a plant cell nuclear genome and is possibly linked to a region encoding constitutive targeted, including phloem expression, which is one means of enabling localization of ASAL protein to whole plant including phloem. The ASAL gene comprises the sequence of (SEQ ID NO: 20)

In fourth construct cassette, the present invention provides a G. hirsutum codon optimized purified DNA construct comprising synthetic Re-PAT synthase protein-encoding region constitutively or localized to a plant cell nuclear genome. In certain embodiments, the Re-PAT gene comprises the sequence of SEQ ID NO: 17.

### Single T-DNA Tetra Gene Creation Method

Three insecticidal Tma12 (SEQ ID NO: 18), PTA (SEQ ID NO: 19), ASAL (SEQ ID NO: 20) and one herbicide gene named Re-Pat (SEQ ID NO: 17) went under codon optimization in a way to make them cotton genome specific. Individual super constitutive promoter with dicot specific un-translated enhancer sequences/regions attached with each gene at its 5′ end along with individual terminator sequence at 3′ end. So, overall it was constituted 5702 bp single T-DNA construct (SEQ ID NO: 21).

### Identification and Isolation of Insecticidal Toxin Genes

The technique articulated in this innovation is expected to be utilized to accomplish enhanced expression of Re-PAT, Tectaria macrodonta Tma12, Pinellia ternata agglutinin (PTA) and Allium satiuvum ASAL as given below. How to identify new insecticidal sequences have described well by Donovan et al 1992, which comprised with following steps: Isolation of tentative insecticidal toxins, amino acid sequencing, back translation, designing of oligonucleotide probe followed by identification and cloning of toxins gene by hybridization. Perlak et al., (1991) used two approaches to increase the toxin levels in genetically modified plants. First those DNA sequences which inhibits excellent plant expression both at translational and mRNA level were selectively removed through side directed mutagenesis to partially modified the gene called (PM) without changing the amino acid sequences. The second one was called fully modified synthetic gene FM. Comparing with wild type, PM sequences had a 10-fold higher expression for insect control while FM sequences had 100-fold higher expression. We used the fully modified gene with GC rich contents to produce higher transcription and translation cellular process in plants including cotton plants.

### Designing of Plant Expression Vectors

Construction of plant expression vectors is done for aiming tissue specific expression of gene comprises constitutive expression specific promoters along with some tissue specific regulator elements like enhancer sequence. Promoters which direct constitutively enhanced expression in plant tissues will be well known to those of skill in the art in light of present discovery. To obtain enhanced constitutive expression, constitutive promoter and enhancer must be attached at 5′ of constitutively expressed gene supplemented with terminator sequence at 3′ of the expressed gene. For example, when Tma12 an insecticidal gene is expressed under Cauliflower Mosaic Virus 35S promoter, it will express in all tissues including phloem. Alternatively, other sources of constitutive promoter may be used for targeting expression of Re-PAT, Tma12, and PTA and ASAL genes.

### Nucleic Acid Composition

In one exemplary embodiment, the tetra gene transgenic pant specifically cotton plant exhibits a novel genotype comprising four expression cassette of transgenes and already incorporated selectable marker gene in the expression vector, the marker gene belongs to the vector use for the transformation.

In the first cassette an appropriate constitutive promoter is attached to a gene that encodes Tma12 protein which confers resistance to transgenic cotton plant tolerant to hemipteran sucking pest range of insects.

A 28 nucleotides long un-translated SynM enhancer sequence is the same sequence of nucleotides of SEQ ID NO: 21 starting from the 7^{th} nucleotide to 34^{th} nucleotide for constitutively directed enhanced expression.

In the second cassette, PTA gene is tagged at 5′end/N-terminal with an appropriate constitutive promoter and same 28 bp enhancer sequence for tagged constitutive expression of Pinellia ternata agglutinin gene in transgenic cotton plant.

The third cassette of this invention comprises ASAL gene in which promoter and enhancer sequences are attached at 5′end for enhanced constitutive directed expression of ASAL gene in the transgenic cotton plant.

In the fourth cassette an appropriate constitutive promoter is attached to a gene that encodes for Re-PAT protein which confers resistance to transgenic cotton plant tolerant to broad and narrow range of weeds.

The already incorporated marker gene into plant expression vector, which when expressed can be used as selection marker. In one embodiment of the present invention the selectable marker gene is kanamycin or hygromycin.

All the transgenes (Tma12, PTA, ASAL and Re-PAT), at their C terminals are linked separately to polyadenylation signals from Agrobacterium tumefaciens nopaline synthase gene (NOS) terminator.

The all four cassette might be inserted into plant on the same or different plasmids.

In a preferred embodiment, the first, second, third and fourth cassettes exist on the same plasmid and are introduced into cotton genome by using Agrobacterium-mediated transformation method. In other embodiments, these genes may be present on different or same T-DNA regions.

In one embodiment, all four cassettes are present on the same T-DNA region.

In a second embodiment, first, second and fourth cassettes are present at same T-DNA region.

In a third embodiment, second, third along with fourth cassettes are present on same T-DNA region.

In a fourth embodiment, first, third and fourth cassettes are present on same T-DNA region.

In a fourth embodiment, first, second and fourth cassettes are present on same T-DNA region.

In a fourth embodiment, first, second and third cassettes are present on same T-DNA region.

### Transformation in Cotton

A very well-known cotton transformation and regeneration procedure present are usually Agrobacterium tumefaciens based mediated transformation of foreign DNA into cotton genome and regeneration of cotton plant parts mostly immature embryos into fully productive genetically modified cotton plants. Mostly dicotyledonous, but some time monocotyledonous plants are transformed by using agrobacterium mediated transformation, but it is more effective against dicotyledonous like cotton plants. The cloning of DNA of interest is done in binary expression vector between left and right T-border consensus sequences, called T-DNA region. The binary vector harboring DNA of interest is transmitted to agrobacterium cell via electroporation method. The electroporated transmitted binary expression vector is then co-cultivated with cotton embryos.

The binary vector comprising the DNA of interest under T-DNA region is then integrated into cotton plant genome. The gene cassettes and selectable marker gene may be present on the same T-DNA regions in the same vector or vice versa.

In one embodiment of the present innovation, the gene cassettes are present on the same T-DNA region.

After transformation, the next step is the selection regeneration of putative transgenic plants via antibiotic drug application to appropriate marker gene (kanamycein or hygromycein) and progeny retaining the foreign DNA. The composition of suitable regeneration medium is well known to any skilled man.

The transgenic plants achieved thus, as described in the present invention, have herbicidal or insecticidal effects. These plants showed tolerance to-non-selective herbicide sprays and are resistant to Hemiptearn sucking pest family comprising Heteroptera, Aleyrodidae, Cicadidae (Aphididae, Adelgidae), Psyllidae, Coccidae and, Eriococcidae which may attack on it. Subsequently, self-defense mechanism is shown by the transgenic cotton plants of the present invention against invasion by sucking pest such as Whitefly (Bemisia tabaci), Aphid (Aphis gossypii) and Jassisd (Amrasca biguttulla). A fewer insecticide sprays are needed for cultivation of invented transgenic cotton plants in comparison to wild-type plants of the same cultivars and minimal loss of yield through insect pest has been observed.

The present innovation is not limited to the aforementioned transgenic cotton plants only but is endorsed comprehensively to take account of any plant material gained from them including seed if at least one of the current invention polynucleotide is contained by them.

The present invention keep plants which are obtained from breeding crosses with the current transgenic cotton plants or resultant there from by orthodox breeding or any other procedure.

The plant material attained from the transgenic plant that may contain additional, changed or fewer polynucleotide sequences matched with genetically modified cotton plants is also covered under this present invention. For example, if someone desires to generate a new event by with the transgenic cotton plant or display other phenotypic features, such as a third insect resistance gene a procedure well-known as gene stacking.

The current innovation also provides methods to obtain higher constitutively targeted expression of Re-PAT, Tma12, PTA and ASAL insecticidal genes in dicotyledonous transgenic plants, without disturbing the normal phenotype and agronomic characteristics of the transgenic plants.

The present invention also allows getting insecticidal toxins at levels up-to 25 times higher than that shown by existing procedures.

The present invention enables transgenic plants expressing Tma12, PTA, ASAL and Re-PAT, gene to be used as an alternative to plants expressing first generation single genes toxins. These next generation toxins with their combined effect will be used both for control as well as resistance management of significant sucking insects range as mentioned above. It is also predicted that three insecticidal toxins having different mode of action in the insect midgut will enhance the effectiveness against target insect pest and will decrease the possibility of developed resistance against these toxin proteins. The higher constitutive expression including phloem tissue will further reduce the chances of insect resistance.

The method of expressing tetra gene—Tma12, PTA, ASAL and Re-PAT, assembled constitutively in cotton plants includes the following steps:
i) Designing and constructing a polynucleotide consisting of a suitable promoter joined to an un-translated enhancer sequence which is further tagged to DNA sequence encoding herbicidal and insecticidal proteins Re-PAT and Tma12, or PTA or ASAL which is further tagged with suitable terminator sequence.
ii) The genes thus tagged with constitutive promoters to express combined proteins and consequently increased combined toxin
iii) Transforming the cotton plants with DNA construct of step (i) so that transgenic plant expresses combined proteins in all tissue of cotton plants.

Any cultivar of dicotyledonous plant including fiber, fruit, legume tuber and any variety of species of monocotyledonous plant is covered by the present invention.

In preferred incarnations, the dicot is a cotton, tomato and potato plant or cell, while maize, rice wheat and sugarcane are preferred embodiments of monocot plant.

### Laboratory Insect Bioassays of Transgenic Plant Events

The identification of transgenic cotton plant expressing high level of Tma12, PTA and ASAL insecticidal proteins of interest and herbicide tolerance, screening is essential of the antibiotic resistant transgenic regenerated plants (T₀ generation) for insecticidal activity and/or expression of interest. Numerous methods well known by those skilled in the art of may help in completion of this task, including but not limited to (1) taking leaf samples from the transgenic T₀ plants and directly going for assay the leaf for activity against susceptible insects in comparison with tissue obtained from a non-transgenic, negative control cotton plant. For example T₀ cotton plants expressing Re-PAT, Tma12, PTA and ASAL can be identified by assaying leaf tissue obtained from such plants for activity against Hemiptearn species. (2) Analysis of extracted protein samples by Enzyme Linked Immuno Sorbent Assay (ELISA) specific for the gene of interest (Tma12, PTA or ASAL): or (3) reverse transcriptase PCR™ to identify events of the expression of genes of interest.

### Method of Expressing Herbicide Re-PAT, and Insecticidal Tma12, PTA and ASAL Gene Proteins in Progeny Plant

The author of this invention further anticipates that the method revealed in this invention comprises a method of generating a transgenic progeny plant. The method of generating such progeny includes: the process of expressing Re-PAT, Tma12, PTA and ASAL herbicidal and insecticidal toxins in a plant disclosed herein includes steps of: (i) Designing and constructing a polynucleotide consisting of suitable constitutive promoter operably joined to a un-translated enhancer sequence which is further tagged to DNA sequence encoding Tma12, PTA, ASAL and Re-PAT, insecticidal and herbicidal proteins which is further linked at 3′ end to a suitable terminator sequence. Thus, these genes attached with subsequent constitutive promoters and enhancers sequences will produce combined toxin proteins. (ii) Procuring a second plant: and (iii) crossing the first and second plants to get crossed transgenic progeny plant that has innate the nucleic acid segments from the first plant. The current innovation precisely includes the progeny plant or seed from any of the transgenic plants, dicot or monocot containing the whole or partial polynucleotide sequence (SEQ ID NO: 21)

### Cloning and Vector Construction

Agrobacterium-mediated transformation vector construction was typically based on employing the restriction digestion and ligation techniques for cloning in sub vectors. The plasmid vector was comprised of the following cassettes: (i) first cassette loaded with Cauliflower mosaic virus (CaMV35S) promoter, un-translated enhancer sequence, a sequence encoding the cotton-optimized Tma12 gene and a NOS polyadenylation terminator sequence; (ii) the second gene cassette consist of Cauliflower mosaic virus (CaMV35S) promoter, dicot specific expression enhancer sequence, a sequence encoding cotton-optimized PTA gene and a NOS polyadenlation terminator sequence (iii) the third gene cassette consist of Cauliflower mosaic virus (CaMV35S) promoter, dicot specific expression enhancer sequence, a sequence encoding cotton-optimized ASAL gene and a NOS polyadenlation terminator sequence (iv) fourth gene cassette containing Cauliflower mosaic virus (CaMV35S) sequence, a un-translated enhancer sequence, a sequence encoding cotton-optimized synthetic Re-PAT gene conferring herbicidal resistance and a NOS polyadenylation sequence; (v) and the already incorporated sequence i.e. marker gene, encoding protein conferring resistance to hygromycin or kanamycin and a NOS polyadenylation sequence.

These gene cassettes were cloned within T-DNA region of vector p4bT3 flanked by left and right border sequences by employing standard Agrobacterium electroporation transformation technique, the above gene constructs is transformed into Agrobacterium tumefaciens strain LB 4404 and the transformed cell culture is selected through kanamycin.

### Cotton Plant Transformation

Regeneration of transgenic cotton plants was done by using standard agrobacterium-mediated transformation method by using germinating embryos of G. hirsutum cv FBS-286 and Eagle-2 as optimized by Ali et al., (2016).

Sterilization of FBS-286 and Eagle-2 delinted seeds was done for 60 seconds by using 10% SDS and 5% Mercuric chloride with enough water covering seeds and continuous shaking of flask. Subsequent washing of seeds was done until no foam was seen. Finally, the washed seeds were further soaked with 10 ml sterilized distilled water. The flask was covered with dark cloth and seeds were allowed to germinate at 30° C. for continuous 36 hours. A 10 ml culture of agrobacterium comprising p4bT3 was grown under suitable antibiotic selections in YEP broth medium. The pellet was dissolved in autoclaved simple MS broth medium after centrifuging it for 10 minutes at 4° C. By removing seed coat and cotyledons tissue germinating seeds were taken out manually. A minor cut towards shoot-apex was given to each isolated embryo with sterilized blade and then put into diluted agrobacterium culture supplemented with acetosyringone (Sigma-Aldrich™) and were allowed to co-cultivate for 1 hour at shaker set at 30° C.

Embryos treated with agrobacterium were blotted on autoclaved filter paper for removing excess bacteria. The embryos were then transformed on petri plates comprising kanamycin and MS medium (MS salt, 4.43 g/L, B5 vitamin, 2 mg/L NAA, 0.1 mg/L kinetin, 30 g/L sucrose, 3.5 g/L Phytogel and 200 mg/ml cefotaxime sodium-salt, pH 5.7). The plates were incubated at in the light at 28° C. for four days after wrapping with paraffin film. The embryos grew in size and turned green. At fifth day, healthy and suspected kanamycin resistant embryos from plates were shifted to 25×200 mm test tubes containing MS media (Same composition as above) supplemented with proper antibiotic again and kept under 14 hours of light and 10 hours at dark conditions for three to four months until healthy shoots were developed. During this period after three weeks shoots were transferred into fresh MS selection free media for rapid growth.

Fully developed and healthy plants were then further transferred to MS medium supplemented with rooting hormones and without antibiotic selection. The plants with healthy roots were then given name putative transgenic plants were shifted to small pots having soil, peat and bhall in certain ratios. The plants were then acclimatized.

### Identification and Selection of Transgenes

The Genomic DNA was extracted from putative transgenic cotton plants and tested through standard polymerase chain reaction techniques by employing gene specific primers sequence (SEQ ID NO: 1-8) for the existence of transgenes (Re-PAT, Tma12, PTA and ASAL).

The positive plant event were identified and went go through screening process of Laboratory insect bioassay for their insecticidal activity against hemiptearn family that is Whitefly (Bemisia tabaci), Aphid (Aphis gossypii) and Jassisd (Amrasca biguttulla) and herbicidal spray in a controlled containments.

### Antibodies Production

Two replication of four male albino rabbits approximately weighing 1.5 kg were intravenously injected at multiple sites separately with purified antigen of Re-PAT, Tma12, PTA and ASAL. The rabbits were fed properly and were injected with respective proteins after further fifteen days. Taking 5 ml blood of each rabbit antibody titer was checked by ELISA. Whole blood was isolated after two month by cardiac puncturing. Serum was stored at −20° C. after isolating with standard procedures. Pre-immune control serum was obtained from rabbits before immunization.

### Antibodies Purification

Rabbit's monoclonal anti-Re-PAT, anti-Tma12, anti-PTA and anti-ASAL antibodies were purified on protein affinity resin. Antibody purified were dialyzed against PBS, dispensed in aliquots and stored frozen at −20° C. ELISA titer was again carried out to check activity of each purified antibody.

### Antibodies Characterization

Protein Extraction

Plant material (leaves, root, and stem) of 200 mg is taken from transgenic as well as non-transgenic cotton plants ground in liquid nitrogen in pre-chilled sterile mortar and pestle. Proper dry ground powder was transferred to 1.5 ml micro tube and was supplemented with 300 μl protein extraction buffer (0.5M EDTA, 0.5M NaCl, 20 mM Tris-HCL pH7.5, 20 mM NH4Cl, 0.5 m PMSF, 10 mM DTT and 0.5M Glycerol). Samples were incubated for one hour-overnight at 4° C. after homogenization by vortexing, and went to centrifugation for 15 minutes at 4° C. at maximum speed. Supernatant was taken, and Bradford reagent extracted protein was quantified on spectrophotometer. For further analysis samples were diluted with 1:10.

Enzyme Linked Immunosorbent Assay

The Re-PAT, Tma12, PTA and ASAL expressed proteins (SEQ ID NO: 22-25) were detected by indirect ELISA. Plant protein samples were denatured in boiling water for 10 minutes and were mixed with 50 mM carbonate buffer (pH, 9.5) and dispensed into 96 well micro titer-plate accordingly and went for incubation at 37° C. three hours to overnight. Tris buffer saline and Tween 20 were used for rinsing unbound antigen. The BSA/TBS blocking buffer (5%) was employed for blocking of unbound non-specific sites and endorsed to bind with anti-Re-PAT, Tma12, PTA and ASAL antibodies respectively.

The bound antibodies were detected by goat anti-rabbit IgG after standard washing using BCIP/NBT substrate. 1N HCL was used to stop the ELISA reaction. Absorbance was taken at 430 nm spectrum, using negative control as blank. Using standard between optical densities of different concentration of standard a graph was plotted. The respective concentrations of Tma12, PTA and ASAL were determined by placing their respective OD values on standard graph. The protein was quantified by using the following formula.

$$\begin{matrix}Transgenic protein \\ ( μ / g leaf tissue )\end{matrix} = Conc . on graph \times \frac{[ 500 \times mg of tissue taken ]}{100} \times dilution factor$$

Immuno Dot Blot

For quick screening of samples having Re-PAT, Tma12, PTA and ASAL expressed proteins of transgenic cotton plants an Immuno Dot Blot analysis was carried out. A tetra gene purified denatured protein samples of transgenic and non-transgenic plants were applied onto nitrocellulose membrane. After drying unbound parts of the membrane were blocked with 5% blocking buffer (BSA/TBS). A primary antibody (anti Re-PAT, Tma12, PTA and ASAL Rabbits 1:10000) was added after washing with thrice with 1×PBS and incubated at 37° C. for one hour. The blot was incubated with secondary IgG (anti-IgG Rabbit mouse AP-conjugated) after given three washing with 1×PBS. After one hour, blot was washed again three times with 1×PBS and BCIP/NBT substrate was added and incubated at 37° C. for 30 minutes for detection of transgenic proteins.

### Genomic DNA Extraction

The total genomic DNA was isolated from leaves of transgenic cotton plants by using CTAB method. A 300 mg sample from leaves was plucked and put immediately into liquid nitrogen container for grinding. Each sample went to fine grinding in pre-chilled Mortar Pestle by using liquid nitrogen. A fresh Eppendorf was loaded with fine ground powder and mixed through with added pre-heated DNA extraction buffer (2% CTAB, 1% Mercapto-ethanol, 2 mM NaCl, 200 mM EDTA, RNase A and 100 mM Tris-HCl).

After incubation at 65° C. for one hour added one volume of phenol (pH: 8), vortexed, spun for 10 minutes at maximum speed. Supernatant was further treated with equal volume of Chloroform:Isoamyalchol (24:1) and spun. After having supernatant again 0.7 volume of isopropanol was added and kept at −20° C. for overnight. After spinning next day pellet was washed twice with 70% chilled ethanol, and re-suspended in 50 μl sterile water after air drying. DNA was quantified on 0.8% agarose gel.

Similarly, Genomic DNA from leaf tissues of Kalgin-5 positive transgenic plant was isolated by using above mentioned protocol to find the event/location junction between transgenic/Gossypium hirstum genome. Gel was run to quantify the Genomic DNA.

### Polymerase Chain Reaction (PCR)

By using gene specific primers (SEQ ID NOS: 1-8) of individual Tma12, PTA and ASAL genes and cassettes (SEQ ID NO: 9-16) PCRs were carried out from isolated genomic. A reaction volume of 25 μl was comprised with 150 ng DNA template, both gene specific primers, 20 picomole each dNTPs mix 3 mM 1× Taq buffer, 2.5 units of Taq Polymerase (Invitrogen). Reaction was carried out in applied Biosciences Thermo cycler with the following conditions: 95° C. for 5 min, (95° C. 35 sec, 55° C. 45 Sec, 72° C. 160 sec)×35 cycles and final extension at 72° C. for 10 minutes and was hold at 20° C. for 5 minutes.

To find the transgenic/cotton genome junction a forward primer SEQ ID NO: 27 is designed from the synthetic sequence of insert at 3′end of the recombinant construct. PCR reaction was made by using the following PCR conditions: 95° C. for 5 min, (95° C. 35 sec, 57° C. 45 Sec, 72° C. 120 sec)×35 cycles and final extension at 72° C. for 10 minutes and was hold at 20° C. for 5 minutes.

### Agarose Gel Electrophoresis

Agarose gel stained with ethidium bromide (0.5 μg/ml) was used for running of PCR amplified gene fragments in 1% TAE buffer. PCR was mixed with 3 μl loading dye (bromo-phenol). Electrophoresis was carried out at 100V for 30 minutes in Gel Electrophoresis apparatus (Bio-Rad) and was observed under UV light in Gel Documentation apparatus (VWR, USA).

A separate gel was run for transgenic/genome junction PCR. PCR product was purified by using Gene JET Gel Extraction kit thermo scientific (K0701). Purified product get sequenced from Macrogen Sequencing Service Korea.

### Transgenic/Genome Junction Polymerase Chain Reaction (PCR)

After getting sequence results of PCR product a primer from the genome of the cotton was designed identified as (SEQ ID NO: 28) and another synthetic primer originated from the T-DNA identified as SEQ ID NO: 27, then by using these both primers a PCR reaction was again run by using the following conditions: PCR reaction was made by using the following PCR conditions: 95° C. for 5 min, (95° C. 35 sec, 56° C. 45 Sec, 72° C. 120 sec)×35 cycles and final extension at 72° C. for 10 minutes and was hold at 20° C. for 5 minutes, and PCR product was run on gel. After again getting it into sequencing a 554 band was achieved.

### Field Test and Observations

After confirming the presence of single T-DNA in transgenic cotton plants subjected to insect bioassay/field test against two main sucking insects/pests whitefly and jassids. A 100% mortality against nymph of both whitefly and jassids was observed along with more than 95% against mature whitefly. Previously, with each single gene maximum mortality rate of whitefly including nymph and mature one was on an average 65%. Single proteins were not effective against Jassids which is the equal lethal sucking pest as whitefly. Due to the synergistic effect of synthetic insecticidal genes higher than 30% more control against nymph & mature whitefly was achieved respectively.

Further, another test related to herbicide was conducted wherein a 1500 ml/acre of herbicide (glufosinate) spray was tolerated by the transgenic cotton plants, which is 700 ml more than the recommendations i.e. 800 ml/acre. In the test, complete weeds destruction was observed at 800 ml/acre.

Table of Sequences

| SEQ | | | |
|---|---|---|---|
| ID | | | |
| NO. | Type | Source | Sequence |
| 1 | DNA | Artificial | ATGTTGATTAGGGATGCTGTTCC |
| | | Sequence | |
| 2 | DNA | Artificial | ATATCAAGCCATCTTCCGAATTT |
| | | Sequence | |
| 3 | DNA | Artificial | CAGTTATGGTTCTTTGTGCTTCTG |
| | | Sequence | |
| 4 | DNA | Artificial | TTAGGTGGTGCTGTGAAGTGAGA |
| | | Sequence | |
| 5 | DNA | Artificial | ACTTTTGCTTTTCTTGCTTCCTG |
| | | Sequence | |
| 6 | DNA | Artificial | CCAATCATTGTTTCTTGAGCAGC |
| | | Sequence | |
| 7 | DNA | Artificial | GTTGAGCAATACAAGTTCATTATGCA |
| | | Sequence | |
| 8 | DNA | Artificial | ATCAACAGTTCCGTTCATAGCCATAA |
| | | Sequence | |
| 9 | DNA | Artificial | CGCTGGAATTCTAGTAGATGCTG |
| | | Sequence | |
| 10 | DNA | Artificial | GCTCTAGCCCTCTCGATAAGTTC |
| | | Sequence | |
| 11 | DNA | Artificial | TCTTCCCTATAACCCAAAGAATCCA |
| | | Sequence | |
| 12 | DNA | Artificial | AAGACCCCTAAACTTGAATCTTCCA |
| | | Sequence | |
| 13 | DNA | Artificial | ACATATGTTCTGAAGAGGGATCTTAC |
| | | Sequence | |
| 14 | DNA | Artificial | CATCTCCATAAAGAACTTGACCAGAA |
| | | Sequence | |
| 15 | DNA | Artificial | CAAATATTGAACACGCTGGAATTCT |
| | | Sequence | |
| 16 | DNA | Artificial | AATTAGTACCAACAGCAACAGC |
| | | Sequence | |
| 17 | DNA | Artificial | ATGTTGATTAGGGATGCTGTTCCTGGAGATTTGCCTGGTA |
| | | Sequence | TTCTTGAAAT CCATAATGAG GCTATTGCTA ACTCTACTGC |
| | | | TATCTGGGAT GAAACACCTG CTGATTTGGA TGAGAGAAGG |
| | | | AGATGGTTCG ATGATAGGAG AGCTAATGGT TTTCCAGTTC |
| | | | TTGTTGCTGA TGTTGATGGT GTTGTTGCTG GATATGCTTC |
| | | | TTACGGAGTT TGGAGGGCTA AGTCTTCATA CAGACATACT |
| | | | GTTGAAAACT CAGTTTACGT TCATGTTGAT CATCATAGGA |
| | | | GAGGTATTGC TACCGCTTTG ATGACTGAAC TTATCGAGAG |
| | | | GGCTAGAGCT GGTGGTATTC ATGTTATCGT TGCTTCTGTT |
| | | | GAATCAACTA ATGCTACATC AGTTGCTTTG CATGAGAGGT |
| | | | TCGGTTTCAG AATCGTTGCT CACATGCCTG AGGTTGGTAG |
| | | | GAAATTCGGA AGATGGCTTG ATATGACATA TCTTCAATTG |
| | | | ACCCTTTGA |
| 18 | DNA | Artificial | ATGGGCAGGA GTTGGGGTGT TGTGGCAGTT ATGGTTCTTT |
| | | Sequence | GTGCTTCTGG TCTTTTGGGT ATCGTTCGTG GTCATGGGTC |
| | | | TATGGAAGAT CCTATTTCTA GGGTTTATAG ATGTAGGCTT |
| | | | GAAAACCCTG AGAGGCCAAC TTCTCCTGCT TGCCAAGCTG |
| | | | CTGTTGCTTT GTCAGGAACA CAGGCTTTCT ACGATTGGAA |
| | | | CGAAGTTAAC ATTCCAAATG CTGCTGGTAG ACATAGGGAG |
| | | | CTTATCCCTG ATGGTCAATT GTGTTCAGCT GGAAGATTCA |
| | | | AGTTTAGGGG TCTTGATTTG GCTAGGTCTG ATTGGATTGC |
| | | | TACCCCTCTT CCATCAGGAG CTTCTTCATT CCCTTTCAGA |
| | | | TATATCGCTA CTGCTGCTCA TTTGGGATTC TTTGAATTTT |
| | | | ACGTTACCAG GGAGGGTTAC CAACCAACTG TTCCTCTTAA |
| | | | GTGGGCTGAT CTTGAAGAGT TGCCTTTTAT TAACGTTACA |
| | | | AATCCTCCAT TGGTTTCTGG ATCATATCAA ATCACCGGTA |
| | | | CTACACCATC TGGTAAATCT GGATCACATC TTATCTATGT |
| | | | TATCTGGCAA AGGACAGATT CACCTGAAGC CTTTTACAGT |
| | | | TGTTCAGATG TTTACTTTAC AGATGCCCTC TCACTTCACA |
| | | | GCACCACCTA A |
| 19 | DNA | Artificial | ATGGCTTCTA AACTTTTGCT TTTCTTGCTT CCTGCTATTT |
| | | Sequence | TGGGTCTTAT TATCCCTAGA CCAGCTGTTG CTGTTGGTAC |
| | | | TAATTATTTG CTTTCAGGAG AAACCTTGGA TACTGATGGT |
| | | | CATCTTAAGA ACGGAGATTT CGATTTTATC ATGCAAGAGG |
| | | | ATTGTAATGC TGTTTTGTAC AATGGTAACT GGCAATCTAA |
| | | | TACTGCTAAC AAGGGAAGAG ATTGCAAATT GACTCTTACA |
| | | | GATAGGGGAG AACTTGTTAT TAATAACGGT GAAGGATCAG |
| | | | CTGTTTGGAG GTCAGGTTCT CAATCAGCTA AAGGAAACTA |
| | | | TGCTGCTGTT TTGCATCCTG AAGGTAAACT TGTTATCTAT |
| | | | GGACCATCTG TTTTCAAAAT CAATCCTTGG GTTCCAGGTC |
| | | | TTAATTCTTT GAGACTTGGA AACGTTCCTT TCACTTGTAA |
| | | | CATGTTGTTT TCTGGTCAAG TTCTTTATGG AGATGGAAAG |
| | | | ATCACAGCTA GGAACCACAT GCTTGTTATG CAAGGAGATT |
| | | | GTAATTTGGT TCTTTACGGT GGAAAGTGCG ATTGGCAATC |
| | | | TAATACACAT GGTAACGGAG AACATTGCTT CTTGAGACTT |
| | | | AACCATAAAG GAGAGTTGAT TATCAAGGAT GATGATTTCA |
| | | | AGTCAATTTG GTCTTCACAA TCTTCATCTA AGCAAGGAGA |
| | | | TTACGTTTTT ATCCTTCAAG ATAATGGTTA TGGTGTTATC |
| | | | TATGGACCAG CTATCTGGGC TACTTCATCT AAGAGGTCAG |
| | | | TTGCTGCTCA AGAAACAATG ATTGGAATGG TTACCGAGAA |
| | | | AGTTAACTGA |
| 20 | DNA | Artificial | ATGGGTCCAA CTACCTCCTC TCCTAAGGCT ATGATGCGTA |
| | | Sequence | TCGCTACCGT GGCTGCTATC CTCACAATCC TCGCTTCAAC |
| | | | CTGTATGGCT AGAAATGTTC TTACAAACGG TGAAGGATTG |
| | | | TATGCTGGAC AATCTCTTGA TGTTGAGCAA TACAAGTTCA |
| | | | TTATGCAAGA TGATTGTAAT CTTGTTTTGT ACGAATACTC |
| | | | TACCCCTATC TGGGCTTCAA ATACCGGTGT TACTGGAAAA |
| | | | AACGGTTGCA GAGCTGTTAT GCAAAGGGAT GGAAACTTCG |
| | | | TTGTTTACGA TGTTAACGGT AGACCAGTTT GGGCTTCTAA |
| | | | TTCAGTTAGG GGTAATGGTA ACTACATTCT TGTTTTGCAA |
| | | | AAGGATAGGA ACGTTGTTAT CTATGGATCT GATATTTGGT |
| | | | CTACTGGTAC ATACAGAAGG TCTGTTGGTG GAGCTGTTGT |
| | | | TATGGCTATG AACGGAACTG TTGATGGTGG ATCAGTTATC |
| | | | GGTCCTGTTG TTGTGAATCA AAAAGATACC GCAGCCATCA |
| | | | GAAAGGTTGG AACTGGGGCA GCCTAA |
| 21 | DNA | Artificial | CCCGGGACAC GCTGGAATTC TAGTATACTA AACCATGGGC |
| | | Sequence | AGGAGTTGGG GTGTTGTGGC AGTTATGGTT CTTTGTGCTT |
| | | | CTGGTCTTTT GGGTATCGTT CGTGGTCATG GGTCTATGGA |
| | | | AGATCCTATT TCTAGGGTTT ATAGATGTAG GCTTGAAAAC |
| | | | CCTGAGAGGC CAACTTCTCC TGCTTGCCAA GCTGCTGTTG |
| | | | CTTTGTCAGG AACACAGGCT TTCTACGATT GGAACGAAGT |
| | | | TAACATTCCA AATGCTGCTG GTAGACATAG GGAGCTTATC |
| | | | CCTGATGGTC AATTGTGTTC AGCTGGAAGA TTCAAGTTTA |
| | | | GGGGTCTTGA TTTGGCTAGG TCTGATTGGA TTGCTACCCC |
| | | | TCTTCCATCA GGAGCTTCTT CATTCCCTTT CAGATATATC |
| | | | GCTACTGCTG CTCATTTGGG ATTCTTTGAA TTTTACGTTA |
| | | | CCAGGGAGGG TTACCAACCA ACTGTTCCTC TTAAGTGGGC |
| | | | TGATCTTGAA GAGTTGCCTT TTATTAACGT TACAAATCCT |
| | | | CCATTGGTTT CTGGATCATA TCAAATCACC GGTACTACAC |
| | | | CATCTGGTAA ATCTGGATCA CATCTTATCT ATGTTATCTG |
| | | | GCAAAGGACA GATTCACCTG AAGCCTTTTA CAGTTGTTCA |
| | | | GATGTTTACT TTACAGATGC CCTCTCACTT CACAGCACCA |
| | | | CCTAAGATCG TTCAAACATT TGGCAATAAA GTTTCTTAAG |
| | | | ATTGAATCCT GTTGCCGGTC TTGCGATGAT TATCATATAA |
| | | | TTTCTGTTGA ATTACGTTAA GCATGTAATA ATTAACATGT |
| | | | AATGCATGAC GTTATTTATG AGATGGGTTT TTATGATTAG |
| | | | AGTCCCGCAA TTATACATTT AATACGCGAT AGAAAACAAA |
| | | | ATATAGCGCG CAAACTAGGA TAAATTATCG CGCGCGGTGT |
| | | | CATCTATGTT ACTAGATCGG GCCCTCAGCG TGTCCTCTCC |
| | | | AAATGAAATG AACTTCCTTA TATAGAGGAA GGTCTTGCGA |
| | | | AGGATAGTGG GATTGTGCGT CATCCCTTAC GTCAGTGGAG |
| | | | ATATCACATC AATCCACTTG CTTTGAAGAC GTGGTTGGAA |
| | | | CGTCTTCTTT TTCCACGATG CTCCTCGTGG GTGGGGGTCC |
| | | | ATCTTTGGGA CCACTGTCGG CAGAGGCATC TTGAACGATA |
| | | | GCCTTTCCTT TATCGCAATG ATGGCATTTG TAGGTGCCAC |
| | | | CTTCCTTTTC TACTGTCCTT TTGATGAAGT GACAGATAGC |
| | | | TGGGCAATGG AATCCGAGGA GGTTTCCCGA TATTACCCTT |
| | | | TGTTGAAAAG TCTCAATAGC CCTTTGGTCT TCTGAGACTG |
| | | | TATCTTTGAT ATTCTTGGAG TAGACGAGAG TGTCGTGCTC |
| | | | CACCATGTTA TCACATCAAT CCACTTGCTT TGAAGACGTG |
| | | | GTTGGAACGT CTTCTTTTTC CACGATGCTC CTCGTGGGTG |
| | | | GGGGTCCATC TTTGGGACCA CTGTCGGCAG AGGCATCTTG |
| | | | AACGATAGCC TTTCCTTTAT CGCAATGATG GCATTTGTAG |
| | | | GTGCCACCTT CCTTTTCTAC TGTCCTTTTG ATGAAGTGAC |
| | | | AGATAGCTGG GCAATGGAAT CCGAGGAGGT TTCCCGATAT |
| | | | TACCCTTTGT TGAAAAGTCT CATTTAAAAC ACGCTGGAAT |
| | | | TCTAGTATAC TAAACCATGG CTTCTAAACT TTTGCTTTTC |
| | | | TTGCTTCCTG CTATTTTGGG TCTTATTATC CCTAGACCAG |
| | | | CTGTTGCTGT TGGTACTAAT TATTTGCTTT CAGGAGAAAC |
| | | | CTTGGATACT GATGGTCATC TTAAGAACGG AGATTTCGAT |
| | | | TTTATCATGC AAGAGGATTG TAATGCTGTT TTGTACAATG |
| | | | GTAACTGGCA ATCTAATACT GCTAACAAGG GAAGAGATTG |
| | | | CAAATTGACT CTTACAGATA GGGGAGAACT TGTTATTAAT |
| | | | AACGGTGAAG GATCAGCTGT TTGGAGGTCA GGTTCTCAAT |
| | | | CAGCTAAAGG AAACTATGCT GCTGTTTTGC ATCCTGAAGG |
| | | | TAAACTTGTT ATCTATGGAC CATCTGTTTT CAAAATCAAT |
| | | | CCTTGGGTTC CAGGTCTTAA TTCTTTGAGA CTTGGAAACG |
| | | | TTCCTTTCAC TTGTAACATG TTGTTTTCTG GTCAAGTTCT |
| | | | TTATGGAGAT GGAAAGATCA CAGCTAGGAA CCACATGCTT |
| | | | GTTATGCAAG GAGATTGTAA TTTGGTTCTT TACGGTGGAA |
| | | | AGTGCGATTG GCAATCTAAT ACACATGGTA ACGGAGAACA |
| | | | TTGCTTCTTG AGACTTAACC ATAAAGGAGA GTTGATTATC |
| | | | AAGGATGATG ATTTCAAGTC AATTTGGTCT TCACAATCTT |
| | | | CATCTAAGCA AGGAGATTAC GTTTTTATCC TTCAAGATAA |
| | | | TGGTTATGGT GTTATCTATG GACCAGCTAT CTGGGCTACT |
| | | | TCATCTAAGA GGTCAGTTGC TGCTCAAGAA ACAATGATTG |
| | | | GAATGGTTAC CGAGAAAGTT AACTGAGATC GTTCAAACAT |
| | | | TTGGCAATAA AGTTTCTTAA GATTGAATCC TGTTGCCGGT |
| | | | CTTGCGATGA TTATCATATA ATTTCTGTTG AATTACGTTA |
| | | | AGCATGTAAT AATTAACATG TAATGCATGA CGTTATTTAT |
| | | | GAGATGGGTT TTTATGATTA GAGTCCCGCA ATTATACATT |
| | | | TAATACGCGA TAGAAAACAA AATATAGCGC |
| | | | GCAAACTAGG |
| | | | ATAAATTATC GCGCGCGGTG TCATCTATGT TACTAGATCT |
| | | | CGCGATCAGC GTGTCCTCTC CAAATGAAAT GAACTTCCTT |
| | | | ATATAGAGGA AGGTCTTGCG AAGGATAGTG GGATTGTGCG |
| | | | TCATCCCTTA CGTCAGTGGA GATATCACAT CAATCCACTT |
| | | | GCTTTGAAGA CGTGGTTGGA ACGTCTTCTT TTTCCACGAT |
| | | | GCTCCTCGTG GGTGGGGGTC CATCTTTGGG ACCACTGTCG |
| | | | GCAGAGGCAT CTTGAACGAT AGCCTTTCCT TTATCGCAAT |
| | | | GATGGCATTT GTAGGTGCCA CCTTCCTTTT CTACTGTCCT |
| | | | TTTGATGAAG TGACAGATAG CTGGGCAATG GAATCCGAGG |
| | | | AGGTTTCCCG ATATTACCCT TTGTTGAAAA GTCTCAATAG |
| | | | CCCTTTGGTC TTCTGAGACT GTATCTTTGA TATTCTTGGA |
| | | | GTAGACGAGA GTGTCGTGCT CCACCATGTT ATCACATCAA |
| | | | TCCACTTGCT TTGAAGACGT GGTTGGAACG TCTTCTTTTT |
| | | | CCACGATGCT CCTCGTGGGT GGGGGTCCAT CTTTGGGACC |
| | | | ACTGTCGGCA GAGGCATCTT GAACGATAGC CTTTCCTTTA |
| | | | TCGCAATGAT GGCATTTGTA GGTGCCACCT TCCTTTTCTA |
| | | | CTGTCCTTTT GATGAAGTGA CAGATAGCTG GGCAATGGAA |
| | | | TCCGAGGAGG TTTCCCGATA TTACCCTTTG TTGAAAAGTC |
| | | | TCATGGCCAA CACGCTGGAA TTCTAGTATA CTAAACCATG |
| | | | GGTCCAACTA CCTCCTCTCC TAAGGCTATG ATGCGTATCG |
| | | | CTACCGTGGC TGCTATCCTC ACAATCCTCG CTTCAACCTG |
| | | | TATGGCTAGA AATGTTCTTA CAAACGGTGA AGGATTGTAT |
| | | | GCTGGACAAT CTCTTGATGT TGAGCAATAC AAGTTCATTA |
| | | | TGCAAGATGA TTGTAATCTT GTTTTGTACG AATACTCTAC |
| | | | CCCTATCTGG GCTTCAAATA CCGGTGTTAC TGGAAAAAAC |
| | | | GGTTGCAGAG CTGTTATGCA AAGGGATGGA AACTTCGTTG |
| | | | TTTACGATGT TAACGGTAGA CCAGTTTGGG CTTCTAATTC |
| | | | AGTTAGGGGT AATGGTAACT ACATTCTTGT TTTGCAAAAG |
| | | | GATAGGAACG TTGTTATCTA TGGATCTGAT ATTTGGTCTA |
| | | | CTGGTACATA CAGAAGGTCT GTTGGTGGAG CTGTTGTTAT |
| | | | GGCTATGAAC GGAACTGTTG ATGGTGGATC AGTTATCGGT |
| | | | CCTGTTGTTG TGAATCAAAA AGATACCGCA GCCATCAGAA |
| | | | AGGTTGGAAC TGGGGCAGCC TAAGATCGTT CAAACATTTG |
| | | | GCAATAAAGT TTCTTAAGAT TGAATCCTGT TGCCGGTCTT |
| | | | GCGATGATTA TCATATAATT TCTGTTGAAT TACGTTAAGC |
| | | | ATGTAATAAT TAACATGTAA TGCATGACGT TATTTATGAG |
| | | | ATGGGTTTTT ATGATTAGAG TCCCGCAATT ATACATTTAA |
| | | | TACGCGATAG AAAACAAAAT ATAGCGCGCA |
| | | | AACTAGGATA |
| | | | AATTATCGCG CGCGGTGTCA TCTATGTTAC TAGATCGCAT |
| | | | GCTCAGCGTG TCCTCTCCAA ATGAAATGAA CTTCCTTATA |
| | | | TAGAGGAAGG TCTTGCGAAG GATAGTGGGA TTGTGCGTCA |
| | | | TCCCTTACGT CAGTGGAGAT ATCACATCAA TCCACTTGCT |
| | | | TTGAAGACGT GGTTGGAACG TCTTCTTTTT CCACGATGCT |
| | | | CCTCGTGGGT GGGGGTCCAT CTTTGGGACC ACTGTCGGCA |
| | | | GAGGCATCTT GAACGATAGC CTTTCCTTTA TCGCAATGAT |
| | | | GGCATTTGTA GGTGCCACCT TCCTTTTCTA CTGTCCTTTT |
| | | | GATGAAGTGA CAGATAGCTG GGCAATGGAA |
| | | | TCCGAGGAGG |
| | | | TTTCCCGATA TTACCCTTTG TTGAAAAGTC TCAATAGCCC |
| | | | TTTGGTCTTC TGAGACTGTA TCTTTGATAT TCTTGGAGTA |
| | | | GACGAGAGTG TCGTGCTCCA CCATGTTATC ACATCAATCC |
| | | | ACTTGCTTTG AAGACGTGGT TGGAACGTCT TCTTTTTCCA |
| | | | CGATGCTCCT CGTGGGTGGG GGTCCATCTT TGGGACCACT |
| | | | GTCGGCAGAG GCATCTTGAA CGATAGCCTT TCCTTTATCG |
| | | | CAATGATGGC ATTTGTAGGT GCCACCTTCC TTTTCTACTG |
| | | | TCCTTTTGAT GAAGTGACAG ATAGCTGGGC AATGGAATCC |
| | | | GAGGAGGTTT CCCGATATTA CCCTTTGTTG AAAAGTCTCA |
| | | | CATATGACAC GCTGGAATTC TAGTATACTA AACCATGTTG |
| | | | ATTAGGGATG CTGTTCCTGG AGATTTGCCT GGTATTCTTG |
| | | | AAATCCATAA TGAGGCTATT GCTAACTCTA CTGCTATCTG |
| | | | GGATGAAACA CCTGCTGATT TGGATGAGAG AAGGAGATGG |
| | | | TTCGATGATA GGAGAGCTAA TGGTTTTCCA GTTCTTGTTG |
| | | | CTGATGTTGA TGGTGTTGTT GCTGGATATG CTTCTTACGG |
| | | | AGTTTGGAGG GCTAAGTCTT CATACAGACA TACTGTTGAA |
| | | | AACTCAGTTT ACGTTCATGT TGATCATCAT AGGAGAGGTA |
| | | | TTGCTACCGC TTTGATGACT GAACTTATCG AGAGGGCTAG |
| | | | AGCTGGTGGT ATTCATGTTA TCGTTGCTTC TGTTGAATCA |
| | | | ACTAATGCTA CATCAGTTGC TTTGCATGAG AGGTTCGGTT |
| | | | TCAGAATCGT TGCTCACATG CCTGAGGTTG GTAGGAAATT |
| | | | CGGAAGATGG CTTGATATGA CATATCTTCA ATTGACCCTT |
| | | | TGAGATCGTT CAAACATTTG GCAATAAAGT TTCTTAAGAT |
| | | | TGAATCCTGT TGCCGGTCTT GCGATGATTA TCATATAATT |
| | | | TCTGTTGAAT TACGTTAAGC ATGTAATAAT TAACATGTAA |
| | | | TGCATGACGT TATTTATGAG ATGGGTTTTT ATGATTAGAG |
| | | | TCCCGCAATT ATACATTTAA TACGCGATAG AAAACAAAAT |
| | | | ATAGCGCGCA AACTAGGATA AATTATCGCG CGCGGTGTCA |
| | | | TCTATGTTAC TAGATCGGTA CC |
| | | | TCAACAAT ATTCCGTCGA CGAGCACGAG CGGAGGACAA |
| | | | TCGATCAAAC ACAAGAAGGA ACAGTGGTGC AAATTTGTTA |
| | | | AGCTTGGCAG GTGCAGCACA ACCGATCACA CAAACCACTA |
| | | | TACCAGTAAA CCTAAGAGAA AAGAGCGAAA |
| | | | ATTGAAAAAG AACCCATTTA AGATATCATC TTTGCCAATC |
| | | | GGAAAACAAA CAAAATTGGG TTATCTGGAT CCCTGCAG |
| 22 | DNA | Artificial | ATGTTGATTAGGGAT GCTGTTCCTGGAGAT TTGCCTGGTAT |
| | | Sequence | TCTT |
| | | | GAAATCCATAATGAG GCTATTGCTAACTCT |
| | | | ACTGCTATCTGGGAT GAAACACCTGCTGAT TTGGATGAGAG |
| | | | AAGG |
| | | | AGATGGTTCGATGAT AGGAGAGCTAATGGT |
| | | | TTTCCAGTTCTTGTT GCTGATGTTGATGGT GTTGTTGCTGG |
| | | | ATAT |
| | | | GCTTCTTACGGAGTT TGGAGGGCTAAGTCT |
| | | | TCATACAGACATACT GTTGAAAACTCAGTT TACGTTCATGT |
| | | | TGAT |
| | | | CATCATAGGAGAGGT ATTGCTACCGCTTTG |
| | | | ATGACTGAACTTATC GAGAGGGCTAGAGCT GGTGGTATTC |
| | | | ATGTT |
| | | | ATCGTTGCTTCTGTT GAATCAACTAATGCT |
| | | | ACATCAGTTGCTTTG CATGAGAGGTTCGGT TTCAGAATCGT |
| | | | TGCT |
| | | | CACATGCCTGAGGTT GGTAGGAAATTCGGA |
| | | | AGATGGCTTGATATG ACATATCTTCAATTG ACCCTTTGA |
| 23 | DNA | Artificial | ATGGGCAGGAGTTGG GGTGTTGTGGCAGTT ATGGTTCTTTG |
| | | Sequence | TGCT |
| | | | TCTGGTCTTTTGGGT ATCGTTCGTGGTCAT |
| | | | GGGTCTATGGAAGAT CCTATTTCTAGGGTT |
| | | | TATAGATGTAGGCTT |
| | | | GAAAACCCTGAGAGG CCAACTTCTCCTGCT |
| | | | TGCCAAGCTGCTGTT GCTTTGTCAGGAACA |
| | | | CAGGCTTTCTACGAT |
| | | | TGGAACGAAGTTAAC ATTCCAAATGCTGCT |
| | | | GGTAGACATAGGGAG CTTATCCCTGATGGT |
| | | | CAATTGTGTTCAGCT |
| | | | GGAAGATTCAAGTTT AGGGGTCTTGATTTG |
| | | | GCTAGGTCTGATTGG ATTGCTACCCCTCTT |
| | | | CCATCAGGAGCTTCT |
| | | | TCATTCCCTTTCAGA TATATCGCTACTGCT |
| | | | GCTCATTTGGGATTC TTTGAATTTTACGTT |
| | | | ACCAGGGAGGGTTAC |
| | | | CAACCAACTGTTCCT CTTAAGTGGGCTGAT |
| | | | CTTGAAGAGTTGCCT TTTATTAACGTTACA |
| | | | AATCCTCCATTGGTT |
| | | | TCTGGATCATATCAA ATCACCGGTACTACA |
| | | | CCATCTGGTAAATCT GGATCACATCTTATC |
| | | | TATGTTATCTGGCAA |
| | | | AGGACAGATTCACCT GAAGCCTTTTACAGT |
| | | | TGTTCAGATGTTTAC TTTACAGATGCCCTC |
| | | | TCACTTCACAGCACC ACCTAA |
| 24 | DNA | Artificial | ATGGCTTCTAAACTT TTGCTTTTCTTGCTT |
| | | Sequence | CCTGCTATTTTGGGT |
| | | | CTTATTATCCCTAGA CCAGCTGTTGCTGTT |
| | | | GGTACTAATTATTTG CTTTCAGGAGAAACC |
| | | | TTGGATACTGATGGT |
| | | | CATCTTAAGAACGGA GATTTCGATTTTATC |
| | | | ATGCAAGAGGATTGT AATGCTGTTTTGTAC |
| | | | AATGGTAACTGGCAA |
| | | | TCTAATACTGCTAAC AAGGGAAGAGATTGC |
| | | | AAATTGACTCTTACA GATAGGGGAGAACTT |
| | | | GTTATTAATAACGGT |
| | | | GAAGGATCAGCTGTT TGGAGGTCAGGTTCT |
| | | | CAATCAGCTAAAGGA AACTATGCTGCTGTT |
| | | | TTGCATCCTGAAGGT |
| | | | AAACTTGTTATCTAT GGACCATCTGTTTTC |
| | | | AAAATCAATCCTTGG GTTCCAGGTCTTAAT |
| | | | TCTTTGAGACTTGGA |
| | | | AACGTTCCTTTCACT TGTAACATGTTGTTT |
| | | | TCTGGTCAAGTTCTT TATGGAGATGGAAAG |
| | | | ATCACAGCTAGGAAC |
| | | | CACATGCTTGTTATG CAAGGAGATTGTAAT |
| | | | TTGGTTCTTTACGGT GGAAAGTGCGATTGG |
| | | | CAATCTAATACACAT |
| | | | GGTAACGGAGAACAT TGCTTCTTGAGACTT |
| | | | AACCATAAAGGAGAG TTGATTATCAAGGAT |
| | | | GATGATTTCAAGTCA |
| | | | ATTTGGTCTTCACAA TCTTCATCTAAGCAA |
| | | | GGAGATTACGTTTTT ATCCTTCAAGATAAT |
| | | | GGTTATGGTGTTATC |
| | | | TATGGACCAGCTATC TGGGCTACTTCATCT |
| | | | AAGAGGTCAGTTGCT GCTCAAGAAACAATG |
| | | | ATTGGAATGGTTACC |
| | | | GAGAAAGTTAACTGA |
| 25 | DNA | Artificial | ATGGGTCCAACTACCTCCTCTCCTAAGGCT |
| | | Sequence | ATGATGCGTATCGCT ACCGTGGCTGCTATC |
| | | | CTCACAATCCTCGCT |
| | | | TCAACCTGTATGGCT AGAAATGTTCTTACA |
| | | | AACGGTGAAGGATTG |
| | | | TATGCTGGACAATCT CTTGATGTTGAGCAA |
| | | | TACAAGTTCATTATG CAAGATGATTGTAAT |
| | | | CTTGTTTTGTACGAA |
| | | | TACTCTACCCCTATC TGGGCTTCAAATACC |
| | | | GGTGTTACTGGAAAA AACGGTTGCAGAGCT |
| | | | GTTATGCAAAGGGAT |
| | | | GGAAACTTCGTTGTT TACGATGTTAACGGT |
| | | | AGACCAGTTTGGGCT TCTAATTCAGTTAGG |
| | | | GGTAATGGTAACTAC |
| | | | ATTCTTGTTTTGCAA AAGGATAGGAACGTT |
| | | | GTTATCTATGGATCT GATATTTGGTCTACT |
| | | | GGTACATACAGAAGG |
| | | | TCTGTTGGTGGAGCT GTTGTTATGGCTATG |
| | | | AACGGAACTGTTGAT GGTGGATCAGTTATC |
| | | | GGTCCTGTTGTTGTG |
| | | | AATCAAAAAGATACC GCAGCCATCAGAAAG |
| | | | GTTGGAACTGGGGCA |
| | | | GCCTAA |
| 26 | DNA | Synthetic | GTTTCAGAATCGTTGCTCACATGCCTGAGGTTGGTAGGAA |
| | | Sequence | ATTCGGAAGATGGCTTGATATGACATATCTTCAATTGACC |
| | | (1-345) + | CTTTGAGATCGTTCAAACATTTGGCAATAAAGTTTCTTAA |
| | | Gossypium | GATTGAATCC TGTTGCCGGT CTTGCGATGA TTATCATATA |
| | | hirsutum | ATTTCTGTTG AATTACGTTA AGCATGTAAT AATTAACATG |
| | | (346-554) | TAATGCATGA CGTTATTTAT GAGATGGGTT TTTATGATTA |
| | | | GAGTCCCGCAATTATACATTTAATACGCGATAGAAAACAA |
| | | | AATATAGCGC GCAAACTAGG ATAAATTATC GCGCGCGGTG |
| | | | TCATCTATGTTACTAGATCGGTACCTCAAC AATATTCCGT |
| | | | CGACGAGCACGAGCGGAGGACAATCGATCA AACACAAGAA |
| | | | GGAACAGTGGTGCAAATTTGTTAAGCTTGGCAGGTGCAGC |
| | | | ACAACCGATC ACACAAACCA CTATACCAGT AAACCTAAGA |
| | | | GAAAAGAGCGAAAATTGAAAAAGAACCCATTTAAGATATC |
| | | | ATCTTTGCCA ATCGGAAAAC AAACAAAATT GGGT |
| 27 | DNA | Artificial | GTTTCAGAATCGTTGCTCACATG |
| | | Sequence | |
| 28 | DNA | Gosspium | ACCCAATTTTGTTTGTTTTCCGA |
| | | hirsutum | |

What is claimed is:

1. A profusion of cassettes having recombinant polynucleotide sequences comprising:
a tetra gene comprising the nucleotide sequence of SEQ ID NO: 21 with a 5′ end attached by a promoter joined to an un-translated enhancer (intron) sequence and a 3′ end attached to a NOS terminator, for encoding the polynucleotide sequences;
wherein the tetra gene comprises sucking pest resistant genes selected from the group consisting of an insecticidal Tma12 gene comprising the nucleotide sequence of SEQ ID NO: 18 from the fern Tectaria macrodonta, a crow dipper gene PTA comprising the nucleotide sequence of SEQ ID NO: 19, an Allium sativum gene ASAL comprising the nucleotide sequence of SEQ ID NO: 20, a Re-PAT gene comprising the nucleotide sequence of SEQ ID NO: 17, and combinations thereof; and
wherein the sucking pest resistant genes are encoded to provide insecticidal and herbicidal toxin proteins in a transgenic plant having constitutively targeted expression, and result in the decreased resistance development against insecticidal toxin proteins and increased efficacy against insect mortality.

2. The profusion of cassettes according to claim 1, wherein a first cassette, a second cassette, a third cassette, and a fourth cassette are located within a T-DNA region of a vector flanked by a left and right border sequence.

3. The profusion of cassettes according to claim 2, wherein the first cassette coding the insecticidal Tma12 gene comprising the nucleotide sequence of SEQ ID NO: 18 operably linked to the promoter, the un-translated enhancer sequence at a 5′ end and the NOS terminator sequence tagged to a 3′ end of the gene Tma12.

4. The profusion of cassettes according to claim 2, wherein the second cassette coding the insecticidal PTA gene comprising the nucleotide sequence of SEQ ID NO: 19 operably linked to the promoter, the un-translated enhancer sequence at a 5′ end and the NOS terminator sequence tagged to a 3′ end of the PTA gene.

5. The profusion of cassettes according to claim 2, wherein the third cassette coding the insecticidal ASAL gene comprising the nucleotide sequence of SEQ ID NO: 20 operably linked to the promoter, the un-translated enhancer sequence at a 5′ end and the NOS terminator sequence tagged to a 3′ end of the ASAL gene.

6. The profusion of cassettes according to claim 2, wherein the fourth cassette coding Re-PAT herbicidal protein gene comprising the nucleotide sequence of SEQ ID NO: 17 operably linked to the promoter, the un-translated enhancer sequence at a 5′ end and the NOS terminator sequence tagged to a 3′ end of Re-PAT gene.

7. The profusion of cassettes according to claim 2, wherein the profusion of cassettes comprising the nucleotide sequence of SEQ ID NO: 21 is present in the transgenic plant or the part of the transgenic plant.

8. The profusion of cassettes according to claim 1, wherein the promoter is Cauliflower mosaic virus (CaMV35S).

9. The profusion of cassettes according to claim 1, wherein the 5′ end of each gene in the transgenic plant is attached with the un-translated enhancer sequence comprising 28 nucleotides of SEQ ID NO. 21 starting from the 7^{th} nucleotide to the 34^{th} nucleotide.

10. The profusion of cassettes according to claim 1, wherein the transgenic plant is a monocot plant selected from the group consisting of maize, sugarcane, and wheat.

11. The profusion of cassettes according to claim 1, wherein the transgenic plant is a dicot plant selected from the group consisting of cotton, potato and tomato.

12. The profusion of cassettes according to claim 11, wherein the dicot plant is the cotton plant.

13. The profusion of cassettes according to claim 1, wherein the profusion of cassettes are located at SEQ ID NO: 26 having a forward primer of SEQ ID NO: 27 and a reverse primer of SEQ ID NO: 28 for identification.

14. A recombinant DNA molecule comprising a polynucleotide sequence selected from the group consisting of SEQ ID NOS: 1-21, SEQ ID NO: 26, SEQ ID NO: 27, and SEQ ID NO: 28 and complements thereof.

15. The recombinant DNA molecule of claim 14, wherein the DNA molecule comprises at least one of SEQ ID NOS: 1-21, SEQ ID NO: 26, SEQ ID NO: 27, SEQ ID NO: 28, and complements thereof in at least a portion of a transgenic cotton plant selected from the group consisting of a cell, seed and part.

16. The recombinant DNA molecule of claim 14, wherein the DNA molecule comprises nucleotide SEQ ID NO: 26 in at least a portion of a transgenic cotton plant selected from the group consisting of a cell, seed and part.

17. The recombinant DNA molecule of claim 14, wherein the DNA molecule comprises at least one of SEQ ID NOS: 1-20 in at least a portion of a transgenic cotton plant selected from the group consisting of a cell, seed and part.

18. The recombinant DNA molecule of claim 14, wherein the DNA molecule comprises at least one of SEQ ID NOS: 17-20 in at least a portion of a transgenic cotton plant selected from the group consisting of a cell, seed and part.

19. The transgenic cotton plant, seed, cells or plant part thereof of claim 15, wherein an amplicon comprises the DNA molecule having the sequence of at least one of SEQ ID NOS: 1-16.

20. The transgenic cotton plant, seed, cells or plant part thereof of claim 15, wherein an amplicon comprises the DNA molecule having the sequence of SEQ ID NO: 21.


SEQUENCE LISTING (32 sequences)

SEQ ID NO: 1 - DNA, 23 bp, Artificial Sequence
  Description of Artificial Sequence Synthetic FBG cotton nucleotide motif
atgttgattagggatgctgttcc

SEQ ID NO: 2 - DNA, 23 bp, Artificial Sequence
  Description of Artificial Sequence Synthetic FBG cotton nucleotide motif
atatcaagccatcttccgaattt

SEQ ID NO: 3 - DNA, 24 bp, Artificial Sequence
  Description of Artificial Sequence Synthetic FBG cotton nucleotide motif
cagttatggttctttgtgcttctg

SEQ ID NO: 4 - DNA, 23 bp, Artificial Sequence
  Description of Artificial Sequence Synthetic FBG cotton nucleotide motif
ttaggtggtgctgtgaagtgaga

SEQ ID NO: 5 - DNA, 23 bp, Artificial Sequence
  Description of Artificial Sequence Synthetic FBG cotton nucleotide motif
acttttgcttttcttgcttcctg

SEQ ID NO: 6 - DNA, 23 bp, Artificial Sequence
  Description of Artificial Sequence Synthetic FBG cotton nucleotide motif
ccaatcattgtttcttgagcagc

SEQ ID NO: 7 - DNA, 26 bp, Artificial Sequence
  Description of Artificial Sequence Synthetic FBG cotton nucleotide motif
gttgagcaatacaagttcattatgca

SEQ ID NO: 8 - DNA, 26 bp, Artificial Sequence
  Description of Artificial Sequence Synthetic FBG cotton nucleotide motif
atcaacagttccgttcatagccataa

SEQ ID NO: 9 - DNA, 23 bp, Artificial Sequence
  Description of Artificial Sequence Synthetic FBG cotton nucleotide motif
cgctggaattctagtagatgctg

SEQ ID NO: 10 - DNA, 23 bp, Artificial Sequence
  Description of Artificial Sequence Synthetic FBG cotton nucleotide motif
gctctagccctctcgataagttc

SEQ ID NO: 11 - DNA, 25 bp, Artificial Sequence
  Description of Artificial Sequence Synthetic FBG cotton nucleotide motif
tcttccctataacccaaagaatcca

SEQ ID NO: 12 - DNA, 25 bp, Artificial Sequence
  Description of Artificial Sequence Synthetic FBG cotton nucleotide motif
aagacccctaaacttgaatcttcca

SEQ ID NO: 13 - DNA, 26 bp, Artificial Sequence
  Description of Artificial Sequence Synthetic FBG cotton nucleotide motif
acatatgttctgaagagggatcttac

SEQ ID NO: 14 - DNA, 26 bp, Artificial Sequence
  Description of Artificial Sequence Synthetic FBG cotton nucleotide motif
catctccataaagaacttgaccagaa

SEQ ID NO: 15 - DNA, 25 bp, Artificial Sequence
  Description of Artificial Sequence Synthetic FBG cotton nucleotide motif
caaatattgaacacgctggaattct

SEQ ID NO: 16 - DNA, 22 bp, Artificial Sequence
  Description of Artificial Sequence Synthetic FBG cotton nucleotide motif
aattagtaccaacagcaacagc

SEQ ID NO: 17 - DNA, 489 bp, Artificial Sequence
  Description of Artificial Sequence Synthetic FBG cotton nucleotide motif
atgttgattagggatgctgttcctggagatttgcctggtattcttgaaatccataatgaggctattgctaactctactgctatctgggatgaaacacctgctgatttggatgagagaaggagatggttcgatgataggagagctaatggttttccagttcttgttgctgatgttgatggtgttgttgctggatatgcttcttacggagtttggagggctaagtcttcatacagacatactgttgaaaactcagtttacgttcatgttgatcatcataggagaggtattgctaccgctttgatgactgaacttatcgagagggctagagctggtggtattcatgttatcgttgcttctgttgaatcaactaatgctacatcagttgctttgcatgagaggttcggtttcagaatcgttgctcacatgcctgaggttggtaggaaattcggaagatggcttgatatgacatatcttcaattgaccctttga

SEQ ID NO: 18 - DNA, 651 bp, Artificial Sequence
  Description of Artificial Sequence Synthetic FBG cotton nucleotide motif
atgggcaggagttggggtgttgtggcagttatggttctttgtgcttctggtcttttgggtatcgttcgtggtcatgggtctatggaagatcctatttctagggtttatagatgtaggcttgaaaaccctgagaggccaacttctcctgcttgccaagctgctgttgctttgtcaggaacacaggctttctacgattggaacgaagttaacattccaaatgctgctggtagacatagggagcttatccctgatggtcaattgtgttcagctggaagattcaagtttaggggtcttgatttggctaggtctgattggattgctacccctcttccatcaggagcttcttcattccctttcagatatatcgctactgctgctcatttgggattctttgaattttacgttaccagggagggttaccaaccaactgttcctcttaagtgggctgatcttgaagagttgccttttattaacgttacaaatcctccattggtttctggatcatatcaaatcaccggtactacaccatctggtaaatctggatcacatcttatctatgttatctggcaaaggacagattcacctgaagccttttacagttgttcagatgtttactttacagatgccctctcacttcacagcaccacctaa

SEQ ID NO: 19 - DNA, 810 bp, Artificial Sequence
  Description of Artificial Sequence Synthetic FBG cotton nucleotide motif
atggcttctaaacttttgcttttcttgcttcctgctattttgggtcttattatccctagaccagctgttgctgttggtactaattatttgctttcaggagaaaccttggatactgatggtcatcttaagaacggagatttcgattttatcatgcaagaggattgtaatgctgttttgtacaatggtaactggcaatctaatactgctaacaagggaagagattgcaaattgactcttacagataggggagaacttgttattaataacggtgaaggatcagctgtttggaggtcaggttctcaatcagctaaaggaaactatgctgctgttttgcatcctgaaggtaaacttgttatctatggaccatctgttttcaaaatcaatccttgggttccaggtcttaattctttgagacttggaaacgttcctttcacttgtaacatgttgttttctggtcaagttctttatggagatggaaagatcacagctaggaaccacatgcttgttatgcaaggagattgtaatttggttctttacggtggaaagtgcgattggcaatctaatacacatggtaacggagaacattgcttcttgagacttaaccataaaggagagttgattatcaaggatgatgatttcaagtcaatttggtcttcacaatcttcatctaagcaaggagattacgtttttatccttcaagataatggttatggtgttatctatggaccagctatctgggctacttcatctaagaggtcagttgctgctcaagaaacaatgattggaatggttaccgagaaagttaactga

SEQ ID NO: 20 - DNA, 546 bp, Artificial Sequence
  Description of Artificial Sequence Synthetic FBG cotton nucleotide motif
atgggtccaactacctcctctcctaaggctatgatgcgtatcgctaccgtggctgctatcctcacaatcctcgcttcaacctgtatggctagaaatgttcttacaaacggtgaaggattgtatgctggacaatctcttgatgttgagcaatacaagttcattatgcaagatgattgtaatcttgttttgtacgaatactctacccctatctgggcttcaaataccggtgttactggaaaaaacggttgcagagctgttatgcaaagggatggaaacttcgttgtttacgatgttaacggtagaccagtttgggcttctaattcagttaggggtaatggtaactacattcttgttttgcaaaaggataggaacgttgttatctatggatctgatatttggtctactggtacatacagaaggtctgttggtggagctgttgttatggctatgaacggaactgttgatggtggatcagttatcggtcctgttgttgtgaatcaaaaagataccgcagccatcagaaaggttggaactggggcagcctaa

SEQ ID NO: 21 - DNA, 5928 bp, Artificial Sequence
  Description of Artificial Sequence Synthetic FBG cotton nucleotide motif
cccgggacacgctggaattctagtatactaaaccatgggcaggagttggggtgttgtggcagttatggttctttgtgcttctggtcttttgggtatcgttcgtggtcatgggtctatggaagatcctatttctagggtttatagatgtaggcttgaaaaccctgagaggccaacttctcctgcttgccaagctgctgttgctttgtcaggaacacaggctttctacgattggaacgaagttaacattccaaatgctgctggtagacatagggagcttatccctgatggtcaattgtgttcagctggaagattcaagtttaggggtcttgatttggctaggtctgattggattgctacccctcttccatcaggagcttcttcattccctttcagatatatcgctactgctgctcatttgggattctttgaattttacgttaccagggagggttaccaaccaactgttcctcttaagtgggctgatcttgaagagttgccttttattaacgttacaaatcctccattggtttctggatcatatcaaatcaccggtactacaccatctggtaaatctggatcacatcttatctatgttatctggcaaaggacagattcacctgaagccttttacagttgttcagatgtttactttacagatgccctctcacttcacagcaccacctaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggccctcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcatttaaaacacgctggaattctagtatactaaaccatggcttctaaacttttgcttttcttgcttcctgctattttgggtcttattatccctagaccagctgttgctgttggtactaattatttgctttcaggagaaaccttggatactgatggtcatcttaagaacggagatttcgattttatcatgcaagaggattgtaatgctgttttgtacaatggtaactggcaatctaatactgctaacaagggaagagattgcaaattgactcttacagataggggagaacttgttattaataacggtgaaggatcagctgtttggaggtcaggttctcaatcagctaaaggaaactatgctgctgttttgcatcctgaaggtaaacttgttatctatggaccatctgttttcaaaatcaatccttgggttccaggtcttaattctttgagacttggaaacgttcctttcacttgtaacatgttgttttctggtcaagttctttatggagatggaaagatcacagctaggaaccacatgcttgttatgcaaggagattgtaatttggttctttacggtggaaagtgcgattggcaatctaatacacatggtaacggagaacattgcttcttgagacttaaccataaaggagagttgattatcaaggatgatgatttcaagtcaatttggtcttcacaatcttcatctaagcaaggagattacgtttttatccttcaagataatggttatggtgttatctatggaccagctatctgggctacttcatctaagaggtcagttgctgctcaagaaacaatgattggaatggttaccgagaaagttaactgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctcgcgatcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcatggccaacacgctggaattctagtatactaaaccatgggtccaactacctcctctcctaaggctatgatgcgtatcgctaccgtggctgctatcctcacaatcctcgcttcaacctgtatggctagaaatgttcttacaaacggtgaaggattgtatgctggacaatctcttgatgttgagcaatacaagttcattatgcaagatgattgtaatcttgttttgtacgaatactctacccctatctgggcttcaaataccggtgttactggaaaaaacggttgcagagctgttatgcaaagggatggaaacttcgttgtttacgatgttaacggtagaccagtttgggcttctaattcagttaggggtaatggtaactacattcttgttttgcaaaaggataggaacgttgttatctatggatctgatatttggtctactggtacatacagaaggtctgttggtggagctgttgttatggctatgaacggaactgttgatggtggatcagttatcggtcctgttgttgtgaatcaaaaagataccgcagccatcagaaaggttggaactggggcagcctaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcatgctcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcacatatgacacgctggaattctagtatactaaaccatgttgattagggatgctgttcctggagatttgcctggtattcttgaaatccataatgaggctattgctaactctactgctatctgggatgaaacacctgctgatttggatgagagaaggagatggttcgatgataggagagctaatggttttccagttcttgttgctgatgttgatggtgttgttgctggatatgcttcttacggagtttggagggctaagtcttcatacagacatactgttgaaaactcagtttacgttcatgttgatcatcataggagaggtattgctaccgctttgatgactgaacttatcgagagggctagagctggtggtattcatgttatcgttgcttctgttgaatcaactaatgctacatcagttgctttgcatgagaggttcggtttcagaatcgttgctcacatgcctgaggttggtaggaaattcggaagatggcttgatatgacatatcttcaattgaccctttgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtacctcaacaatattccgtcgacgagcacgagcggaggacaatcgatcaaacacaagaaggaacagtggtgcaaatttgttaagcttggcaggtgcagcacaaccgatcacacaaaccactataccagtaaacctaagagaaaagagcgaaaattgaaaaagaacccatttaagatatcatctttgccaatcggaaaacaaacaaaattgggttatctggatccctgcag

SEQ ID NO: 22 - DNA, 489 bp, Artificial Sequence; identical to SEQ ID NO: 17
  Description of Artificial Sequence Synthetic FBG cotton nucleotide motif
  CDS (1)..(486)

SEQ ID NO: 23 - DNA, 651 bp, Artificial Sequence; identical to SEQ ID NO: 18
  Description of Artificial Sequence Synthetic FBG cotton nucleotide motif
  CDS (1)..(648)

SEQ ID NO: 24 - DNA, 810 bp, Artificial Sequence; identical to SEQ ID NO: 19
  Description of Artificial Sequence Synthetic FBG cotton nucleotide motif
  CDS (1)..(807)

SEQ ID NO: 25 - DNA, 546 bp, Artificial Sequence; identical to SEQ ID NO: 20
  Description of Artificial Sequence Synthetic FBG cotton nucleotide motif
  CDS (1)..(543)

SEQ ID NO: 26 - DNA, 554 bp, Artificial Sequence
  Description of Artificial Sequence Synthetic insert sequence and Gossypium hirsutum
gtttcagaatcgttgctcacatgcctgaggttggtaggaaattcggaagatggcttgatatgacatatcttcaattgaccctttgagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggtacctcaacaatattccgtcgacgagcacgagcggaggacaatcgatcaaacacaagaaggaacagtggtgcaaatttgttaagcttggcaggtgcagcacaaccgatcacacaaaccactataccagtaaacctaagagaaaagagcgaaaattgaaaaagaacccatttaagatatcatctttgccaatcggaaaacaaacaaaattgggt

SEQ ID NO: 27 - DNA, 23 bp, Artificial Sequence
  Description of Artificial Sequence Synthetic primer sequence from inserted DNA
gtttcagaatcgttgctcacatg

SEQ ID NO: 28 - DNA, 23 bp, Artificial Sequence
  Description of Artificial Sequence Synthetic primer sequence from Gossypium hirsutum Genome
acccaattttgtttgttttccga

SEQ ID NO: 29 - protein, 162 aa, Artificial Sequence
  Description of Artificial Sequence Synthetic FBG cotton motif
MLIRDAVPGDLPGILEIHNEAIANSTAIWDETPADLDERRRWFDDRRANGFPVLVADVDGVVAGYASYGVWRAKSSYRHTVENSVYVHVDHHRRGIATALMTELIERARAGGIHVIVASVESTNATSVALHERFGFRIVAHMPEVGRKFGRWLDMTYLQLTL

SEQ ID NO: 30 - protein, 216 aa, Artificial Sequence
  Description of Artificial Sequence Synthetic FBG cotton motif
MGRSWGVVAVMVLCASGLLGIVRGHGSMEDPISRVYRCRLENPERPTSPACQAAVALSGTQAFYDWNEVNIPNAAGRHRELIPDGQLCSAGRFKFRGLDLARSDWIATPLPSGASSFPFRYIATAAHLGFFEFYVTREGYQPTVPLKWADLEELPFINVTNPPLVSGSYQITGTTPSGKSGSHLIYVIWQRTDSPEAFYSCSDVYFTDALSLHSTT

SEQ ID NO: 31 - protein, 269 aa, Artificial Sequence
  Description of Artificial Sequence Synthetic FBG cotton motif
MASKLLLFLLPAILGLIIPRPAVAVGTNYLLSGETLDTDGHLKNGDFDFIMQEDCNAVLYNGNWQSNTANKGRDCKLTLTDRGELVINNGEGSAVWRSGSQSAKGNYAAVLHPEGKLVIYGPSVFKINPWVPGLNSLRLGNVPFTCNMLFSGQVLYGDGKITARNHMLVMQGDCNLVLYGGKCDWQSNTHGNGEHCFLRLNHKGELIIKDDDFKSIWSSQSSSKQGDYVFILQDNGYGVIYGPAIWATSSKRSVAAQETMIGMVTEKVN

SEQ ID NO: 32 - protein, 181 aa, Artificial Sequence
  Description of Artificial Sequence Synthetic FBG cotton motif
MGPTTSSPKAMMRIATVAAILTILASTCMARNVLTNGEGLYAGQSLDVEQYKFIMQDDCNLVLYEYSTPIWASNTGVTGKNGCRAVMQRDGNFVVYDVNGRPVWASNSVRGNGNYILVLQKDRNVVIYGSDIWSTGTYRRSVGGAVVMAMNGTVDGGSVIGPVVVNQKDTAAIRKVGTGAA